(19) United States
(12) Patent Application Publication
(10) Pub. No.: US 2023/0008034 A1
(43) Pub. Date: Jan. 12, 2023

(54) METHOD FOR MANUFACTURING THIN-WALLED MOLDED ARTICLE, AND WELL PLATE

(71) Applicant: St. Luke's International University, Chuo-ku, Tokyo (JP)

(72) Inventors: Shinya YAMAHIRA, Chuo-ku, Tokyo (JP); Yuji HEIKE, Chuo-ku, Tokyo (JP)

(21) Appl. No.: 17/780,650

(22) PCT Filed: Nov. 26, 2020
(86) PCT No.: PCT/JP2020/044018

§ 371 Date: May 27, 2022

(30) Foreign Application Priority Data
Nov. 29, 2019 (JP) 2019-216608

(51) Int. Cl. B29C 59/02; B29C 33/38; B01L 3/00; B21D 22/02

(52) U.S. Cl. CPC B29C 59/022; B29C 33/3842; B01L 3/5085; B21D 22/02; B29K 2883/00; B29K 2995/0039; B29K 2995/0046; B29L 2031/712

(57) ABSTRACT

The present invention has: a step for heating a resin or a metal in a state in which the resin or metal is sandwiched by a mold provided with a projection that is constituted from an elastic body having a heat resistance temperature higher than the temperature at which the resin or the metal softens, and a support body which is larder than the mold and which is softened by heat at a higher temperature than the resin or the metal, while a force is applied to the mold in the direction toward the resin or the metal.

### TECHNICAL FIELD

The present invention relates to a method for producing a thin-walled molded article having a thin portion in a part of its shape, and a well plate.

### BACKGROUND ART

A technique of molding a resin or the like into a thin structure or film is a very important technique in the development and production of parts of mobile phones and personal computers, other precision equipment, and the like. In particular, in recent years, in high-value-added products used in advanced research and medical care, a method of molding a resin or the like into a thin structure has an increased importance. For example, a container for PCR used for amplifying nucleic acid in basic research or medical tests is designed to have a small thickness in order to efficiently conduct heat into the container. In addition, a PCR container having a thin and flat bottom that enables PCR to be performed after analyzing cells with a microscope is also sold.

In addition, in microscopic observation, the focal length of the objective lens becomes shorter as the magnification becomes higher, which requires that the bottom of the container for high magnification observation should be thin. Conventionally, as such a container for high magnification observation, a product in which a thin cover glass or film is attached to the bottom of the container with an adhesive or the like has been sold, but to avoid the problem of elution of the adhesive and to improve the container strength, a product in which the container and the bottom are integrally molded came to be sold.

A method for producing a thin molded product is a highly demanded technique. Conventionally, the following methods have been reported: a method using a complex molding apparatus incorporating many sensors and metal movable parts (see, for example, Patent Literatures 1 and 2); a method using a compound with which a thin molded product can be easily produced (see, for example, Patent Literature 3.); and a method in which strict conditions are set (see, for example, Patent Literature 4).

### CITATION LIST

### Patent Literature

Patent Literature 1: JP 3767465 B2

Patent Literature 2: JP 2837335 B2

Patent Literature 3: WO 2007/055305 A

Patent Literature 4: JP H09-262883 A

### SUMMARY OF INVENTION

### Problem to be Solved

However, these methods often require very advanced processing techniques and expensive equipment, and the materials are often limited. In general, a molding method such as injection molding or press molding using a metal mold (die) is used for molding a product made of a thermoplastic material such as a resin. However, in injection molding, it is very difficult to cause molten resin to flow into an extremely thin portion of a die. In addition, in injection molding, press molding, and other molding methods, a die that is extremely highly designed and produced with high accuracy, with deformation due to thermal expansion being taken into consideration, as well as an advanced molding process are required in order to, for example, perform resin processing of a thickness of 50 μm or less with a small error over a wide range of several tens of square centimeters or more. In addition to such technical difficulties, it usually takes a very high cost of one million to tens of millions of yen to produce a die, and thus, the development and production of such a high-value-added product require a high technology and a large amount of cost. There is also a method of bonding a thin film instead of molding the thin portion, but this involves not only a problem of complication of the producing process but also problems such as elution of a bonding adhesive and a decrease in structural strength. Therefore, it is desirable to perform integral molding.

Usually, a mold used for molding a resin or the like is made of a rigid material for reproducibly producing a molded product. However, in order to mold a structure having a thickness of several tens of μm over the entirety of a molded product of several tens of square centimeters or at each point thereof, there are the following problems: it is necessary to produce a precise mold having no distortion over a wide range, and no error is allowed even in the molding process. This is because the thickness at each point of the molded product greatly changes due to some errors in the mold and the molding process, and furthermore, the mold breaks through the resin that is the base of the molded product to make a hole. Therefore, a method for simply and inexpensively molding a structure having a precise thin portion is strongly desired in the development and production of various precision equipment as well as research and medical devices.

The present invention has been made in view of the above problems, and an object thereof is to provide a method for producing a thin-walled molded article and a well plate capable of easily and inexpensively molding a thin shape and molding a thin structure over a wide area with a small error.

### Means to Solve the Problem

A method for producing a thin-walled molded article according to the first aspect of the present invention includes: a step of heating a resin or a metal in a state of being interposed between a mold and a support in such a manner that a force in a direction to the resin or the metal is applied to the mold, wherein the mold is provided with a protrusion formed of an elastic body having a heat resistant temperature higher than a softening temperature of the resin or the metal, and the support is harder than the mold and is softened by heat at a temperature higher than a temperature for the resin or the metal; and a step of removing the mold.

According to this configuration, the resin or the metal softened by heat is sufficiently softer than the protrusion of the mold at the initial stage of the step, and therefore the resin or the metal is deformed by being pressed against the mold, whereby a recess is formed. When the thickness of the resin or the metal is sufficient, the force required to reduce the thickness of the bottom of the recess is mainly a force required to deform the resin or the metal. On the other hand, as the step proceeds and the thin portion at the bottom of the recess becomes thinner, the force required to further reduce the thickness of the bottom of the recess is mainly a force required for discharging the resin or the metal from the thin portion against the frictional force acting between the mold or the support and the resin or the metal, rather than the force required to deform the resin or the metal. This force increases dramatically as the thickness of the thin portion decreases. In addition, since the thin portion is less likely to be deformed, the stress applied to the mold increases, and the protrusion of the mold formed of the elastic body is crushed and deformed thereby expanding the area of the thin portion. At this time, the thin portion has a nearly flat shape. Since the frictional force acting between the resin or the metal and the mold or the support further increases due to the increase in the area of the thin portion, as the thin portion of the bottom of the recess becomes thinner, the force required to further reduce the thickness of the bottom of the depression increases at an accelerated rate. Therefore, even if an error occurs in the pressure to the thin portion at each point of a workpiece due to the difference in the shape of each protrusion of the mold and the non-precision of the pressurization process, this pressure error is a very small force with respect to the pressure required to change the thickness of the thin portion that has become sufficiently thin. This allows a shape in which a recess has a thin wall to be easily and inexpensively molded, and allows a structure in which a recess has a thin wall to be molded over a wide area with a small error. As a result, it is possible to produce a thin-walled molded article in which a recess has a flat bottom and has a small bottom thickness (has a thin bottom wall). This allows a shape in which a recess has a thin wall to be easily and inexpensively molded, and allows a structure in which a recess has a thin wall to be molded over a wide area with a small error. Besides, a thicker shape and a thinner shape can be integrally molded. Further, a thicker shape and a thinner shape can be continuously formed with a curved surface. In addition, a technique capable of forming an extremely thin structure having a wall thickness on the order of 10 μm has not been reported, and the present technique is also excellent in precision.

A method for producing a thin-walled molded article according to a second aspect of the present invention is the method for producing a thin-walled molded article according to the first aspect, in which the resin is an amorphous plastic having a glass transition point lower than a heat resistant temperature of an elastic body of the mold, or a crystalline plastic having a melting point lower than the heat resistant temperature of the elastic body of the mold.

According to this configuration, the resin softened by heat is sufficiently softer than the protrusion of the mold at the initial stage of the step, and therefore the resin is deformed by being pressed against the mold, whereby a recess is formed. When the thickness of the resin is sufficient, the force required to reduce the thickness of the bottom of the recess is mainly a force required to deform the resin. On the other hand, as the step proceeds and the thin portion at the bottom of the recess becomes thinner, the force required to further reduce the thickness of the bottom of the recess is mainly a force required for discharging the resin from the thin portion against the frictional force acting between the mold or the support and the resin, rather than the force required to deform the resin. This force increases dramatically as the thickness of the thin portion decreases. In addition, since the thin portion is less likely to be deformed, the stress applied to the mold increases, and the protrusion of the mold formed of the elastic body is crushed and deformed thereby expanding the area of the thin portion. At this time, the thin portion has a nearly flat shape. Since the frictional force acting between the resin and the mold or the support further increases due to the increase in the area of the thin portion, as the thin portion of the bottom of the recess becomes thinner, the force required to further reduce the thickness of the bottom of the depression increases at an accelerated rate. Therefore, even if an error occurs in the pressure to the thin portion at each point of a workpiece due to the difference in the shape of each protrusion of the mold and the non-precision of the pressurization process, this pressure error is a very small force with respect to the pressure required to change the thickness of the thin portion that has become sufficiently thin. This allows a shape in which a recess has a thin wall to be easily and inexpensively molded, and allows a structure in which a recess has a thin wall to be molded over a wide area with a small error.

A method for producing a thin-walled molded article according to a third aspect of the present invention is the method for producing a thin-walled molded article according to the first aspect, in which the metal has a melting point lower than a heat resistant temperature of an elastic body of the mold.

According to this configuration, the metal softened by heat is sufficiently softer than the protrusion of the mold at the initial stage of the step, and therefore the metal is deformed by being pressed against the mold, whereby a recess is formed. When the thickness of the metal is sufficient, the force required to reduce the thickness of the bottom of the recess is mainly a force required to deform the metal. On the other hand, as the step proceeds and the thin portion at the bottom of the recess becomes thinner, the force required to further reduce the thickness of the bottom of the recess is mainly a force required for discharging the metal from the thin portion against the frictional force acting between the mold or the support and the metal, rather than the force required to deform the metal. This force increases dramatically as the thickness of the thin portion decreases. In addition, since the thin portion is less likely to be deformed, the stress applied to the mold increases, and the protrusion of the mold formed of the elastic body is crushed and deformed thereby expanding the area of the thin portion. At this time, the thin portion has a nearly flat shape. Since the frictional force acting between the metal and the mold or the support further increases due to the increase in the area of the thin portion, as the thin portion of the bottom of the recess becomes thinner, the force required to further reduce the thickness of the bottom of the depression increases at an accelerated rate. Therefore, even if an error occurs in the pressure to the thin portion at each point of a workpiece due to the difference in the shape of each protrusion of the mold and the non-precision of the pressurization process, this pressure error is a very small force with respect to the pressure required to change the thickness of the thin portion that has become sufficiently thin. This allows a shape in which a recess has a thin wall to be easily and inexpensively molded, and allows a structure in which a recess has a thin wall to be molded over a wide area with a small error.

A method for producing a thin-walled molded article according to a fourth aspect of the present invention is the method for producing a thin-walled molded article according to the first or second aspect, in which the resin is a thermoplastic resin.

According to this configuration, a thin shape can be easily and inexpensively molded. In addition, a thin structure can be molded over a wide area with a small error.

A method for producing a thin-walled molded article according to a fifth aspect of the present invention is the method for producing a thin-walled molded article according to any one of the first to fourth aspects, the method including: a step of producing a jig having a through hole, provided with a thin film of an elastic body on its front surface; a step of sucking the thin film from a back surface side through the through hole; a step of injecting a solution of an elastic body from above the thin film deflected after the sucking; and a step of forming the mold by heating and curing the solution.

According to this configuration, it is possible to produce a mold provided with a protrusion made of an elastic body. By heating this mold in a state in which the surface provided with the protrusion is in contact with a resin or a metal that is deformed by heat at a temperature lower than a heat resistant temperature of the mold, a thin-walled molded article in which a recess has a flat bottom and has a small bottom thickness can be produced.

A method for producing a thin-walled molded article according to a sixth aspect of the present invention is the method for producing a thin-walled molded article according to any one of the first to fifth aspects, in which the elastic body is polydimethylsiloxane (PDMS).

With this configuration, a shape in which a recess has a thin wall can be easily and inexpensively molded, and a structure in which a recess has a thin wall can be molded over a wide area with a small error.

A method for producing a thin-walled molded article according to a seventh aspect of the present invention is the method for producing a thin-walled molded article according to any one of the first to sixth aspects, in which the thin-walled molded article is a well plate.

With this configuration, a shape in which a recess has a thin wall can be easily and inexpensively molded, and a structure in which a recess has a thin wall can be molded over a wide area with a small error.

The well plate according to an eighth aspect of the present invention is a well plate formed with a resin and provided with at least one well, in which the well has a round bottom, and a bottom central portion of the well has a thickness of 200 μm or less.

According to this configuration, the well has a round bottom, whereby cells seeded in the well gather at the center of the bottom of the well, which facilitates the observation of the cells. In addition, since the thickness of the bottom central portion of the well is 200 μm or less, this enables the observation with a high magnification microscope.

A well plate according to a ninth aspect of the present invention is the well plate according to the eighth aspect, in which a ratio obtained by dividing a radius of curvature of a well bottom by a radius of the well is 0.7 to 1.5.

According to this configuration, the well has a round bottom, whereby cells seeded in the well gather at the center of the bottom of the well, which facilitates the observation of the cells.

A well plate according to a ninth aspect of the present invention is the well plate according to the eighth aspect, in which a ratio obtained by dividing a radius of curvature of the well bottom by a radius of the well is 0.7 to 1.5.

According to this configuration, the range of the shape of the well bottom is defined.

A well plate according to a tenth aspect of the present invention is the well plate according to the eighth or ninth aspect, in which, when a ratio obtained by dividing the radius of curvature of the well bottom by the radius of the well is given as “y”, and a bottom central portion of the well is given as “x” [μm], when the bottom central portion of the well is in a range of 7 to 19 μm, the ratio y satisfies y=−0.0093x+0.9924 or is within ±10% of this value, and when the bottom central portion of the well is in a range of 19 to 200 μm, the ratio y satisfies y=0.0028x+0.7572 or is within ±10% of this value.

According to this configuration, the range of the shape of the well bottom is defined.

A well plate according to an eleventh aspect of the present invention is the well plate according to any one of the eighth to tenth aspects, in which the bottom central portion of the well has an average thickness of 7 μm or more.

According to this configuration, it is possible to realize a well plate having such a strength that most of the wells cannot be damaged.

A well plate according to a twelfth aspect of the present invention is the well plate according to any one of the eighth to eleventh aspects, in which the bottom central portion of the well has a thickness of 150 μm or less.

According to this configuration, the well has a round bottom, whereby cells seeded in the well gather at the center of the bottom of the well, which facilitates the observation of the cells. Furthermore, the thickness of bottom central portion of the well is 150 μm or less, which enables the focus to be adjusted to the cells present on the well bottom even with an oil immersion 100× objective lens, whereby detailed microscopic analysis of a small number, ranging from one, of cells can be reliably performed.

### Effects of Invention

According to one aspect of the present invention, a thin shape can be easily and inexpensively molded. In addition, a thin structure can be molded over a wide area with a small error. Besides, a thicker shape and a thinner shape can be integrally molded. Further, a thicker shape and a thinner shape can be continuously formed with a curved surface. In addition, a press molding technique capable of forming an extremely thin structure having a wall thickness on the order of 10 μm has not been reported, and the present technique is also excellent in precision.

### BRIEF DESCRIPTION OF DRAWINGS

FIG. 1 is a schematic view showing schematic steps of a method for producing a well plate according to an embodiment of the present invention.

FIG. 2 is a flowchart showing an exemplary flow of the method for producing a well plate according to the embodiment of the present invention.

FIG. 3 is an exploded perspective view for explaining attachment of a PDMS thin film to a jig having through holes.

FIG. 4 is a graph showing exemplary experimental results regarding the relationship between the suction pressure and the change distance of the center of the PDMS thin film.

FIG. 5 is a schematic diagram for explaining a method for producing a PDMS mold.

FIG. 6(a) is an overall image of a PDMS mold photographed from above with a stereomicroscope. FIG. 6(b) is a magnified image of the PDMS mold photographed from above with a stereomicroscope. FIG. 6(c) is a magnified image of the PDMS mold photographed at a diagonal angle from above with a stereomicroscope. FIG. 6(d) is a magnified image of the PDMS mold photographed at another diagonal angle from above with a stereomicroscope.

FIG. 7(a) is an overall image of a molded product photographed from above with a stereomicroscope. FIG. 7(b) is a magnified image of a PDMS mold photographed from above with a stereomicroscope. FIG. 7(c) is a magnified image of a PDMS mold photographed at a diagonal angle from above with a stereomicroscope. FIG. 7(d) is a magnified image of a PDMS mold photographed at another diagonal angle from above with a stereomicroscope.

FIG. 8(a) is an overall image of bright field observation of well bottoms according to Example 1 using a microscope. FIG. 8(b) is a magnified image of bright field observation of a well bottom of one well according to Example 1 using a microscope.

FIG. 9(a) is a graph showing the distribution of protrusion heights of the PDMS mold according to Example 1. FIG. 9(b) is a graph showing the distribution of well bottom thicknesses of the molded product according to Example 1. FIG. 9(c) is a graph showing the distribution of well depths of the molded product according to Example 1.

FIG. 10(a) is a cross-sectional observation image of a protrusion of the PDMS mold according to Example 1. FIG. 10(b) is a cross-sectional observation image of an entire molded product according to Example 1. FIG. 10(c) is a cross-sectional observation image of a well bottom of the molded product according to Example 1.

FIG. 11(a) is an overall image of bright field observation of well bottoms according to Example 2 using a microscope. FIG. 11(b) is a magnified image of bright field observation of a well bottom of one well according to Example 2 using a microscope.

FIG. 12(a) is a graph showing the distribution of protrusion heights of the PDMS mold according to Example 2. FIG. 12(b) is a graph showing the distribution of well bottom thicknesses of the molded product according to Example 2. FIG. 12(c) is a graph showing the distribution of well depths of the molded product according to Example 2.

FIG. 13 is a cross-sectional observation image of a well bottom of the molded product according to Example 2.

FIG. 14 shows exemplary Ba/F3 cells imaged using an oil immersion 100× objective lens in cases of various bottom thicknesses.

FIG. 15 is a diagram showing an example of characteristic damage that increases depending on the well bottom thinness.

FIG. 16 is a table showing exemplary experimental results regarding the relationship between the average thickness of the well bottom thinnest portion and the damaged well rate in 384 wells.

FIG. 17 is an exemplary image of a longitudinal section of a well obtained by a confocal microscope.

FIG. 18 shows exemplary images of longitudinal sections of wells obtained by a confocal microscope in cases of various bottom thicknesses.

FIG. 19 is a schematic diagram for explaining a difference in the radius of curvature due to a difference in the thickness t of the well bottom thinnest portion.

FIG. 20 is a graph showing experimental results regarding the relationship between the thickness of the well bottom thinnest portion and the ratio obtained by dividing the radius of curvature of the well bottom by the radius of the well.

### DESCRIPTION OF EMBODIMENTS

Hereinafter, each embodiment is described with reference to the drawings. However, unnecessarily detailed description may be omitted. For example, a detailed description of a well-known matter and a repeated description of substantially the same configuration may be omitted. This is to avoid unnecessary redundancy of the following description and to facilitate understanding of those skilled in the art.

### First Embodiment

In the present embodiment, it has been devised not to precisely construct a mold or a molding process but to add a shape correction function to the mold itself. That is, in the present embodiment, at least the portion of the protrusion of the mold is made of a material that is flexibly deformed (that is, an elastic body), whereby the mold is deformed so that the respective thin portions of the material that is the base of the molded product have identical thicknesses, and a thin structure can be molded with a small error even over a large area.

In the present embodiment, a well plate is exemplified as an example of a thin-walled molded article having a thin portion in a part of the shape, and a method for producing the well plate is described. In the present embodiment, a thin-walled molded article (here, a well plate as an example) to be molded may be referred to as a molded product.

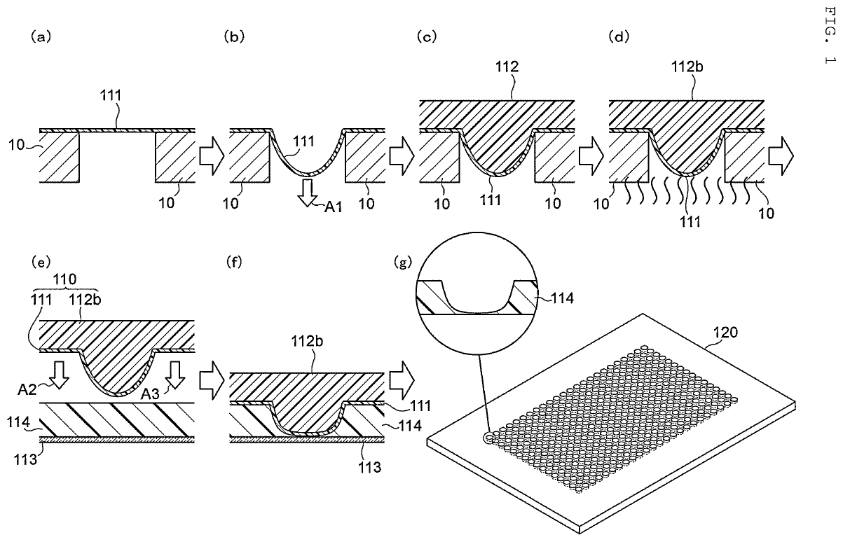

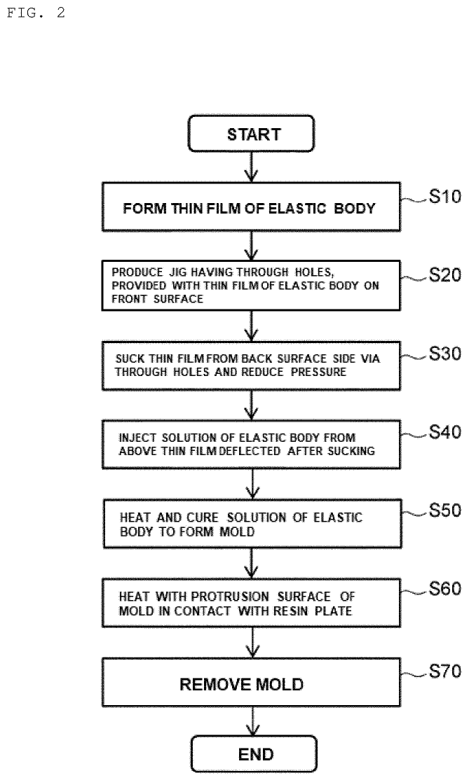

A method for producing a well plate according to the present embodiment is described using FIG. 2 while referring to FIG. 1. FIG. 1 is a schematic view showing schematic steps of a method for producing a well plate according to the present embodiment. FIG. 2 is a flowchart showing an exemplary flow of the method for producing a well plate according to the present embodiment.

(Step S10) First, a thin film of an elastic body is formed. Here, the elastic body is, for example, polydimethylsiloxane (PDMS), and a PDMS thin film is formed.

(Step S20) Next, a jig with having through holes, provided with a thin film of an elastic body on its front surface, is prepared using the thin film (for example, a PDMS thin film) formed in step S10. In the jig having through holes, for example, through holes are provided in an array of 24 columns×16 rows so as to correspond to the holes of the well plate. As a result, as illustrated in the partial cross-sectional view of FIG. 1(a), a PDMS thin film 111 is formed on a front surface of a jig 10 with through holes.

(Step S30) Next, as shown in the partial cross-sectional view shown in FIG. 1(b), the PDMS thin film is sucked and/or depressurized at a set pressure through the through holes from the back surface side of the jig 10 with through holes. As a result, the PDMS thin film is bent in the direction of the arrow A1 in FIG. 1(b). Here, as an example, the thin film is sucked by the pressure reduction.

(Step S40) Next, as shown in the partial cross-sectional view shown in FIG. 1(c), a solution 112 of an elastic body (for example, a PDMS solution) is injected from above the PDMS thin film bent after sucking.

(Step S50) Next, as shown in the partial cross-sectional view shown in FIG. 1(d), the solution 112 of the elastic body (for example, a PDMS solution) is heated and cured, whereby a mold (for example, a PDMS mold) is formed. In this way, in a state in which the PDMS thin film is deformed into convex shapes, a material serving as a mold (for example, PDMS) is injected and cured, whereby a PDMS cured layer 112b is formed. Thus, a mold 110 having protrusions in an array of, for example, 24 columns×16 rows is formed by PDMS. In the mold 110, the PDMS cured layer 112b is formed on the PDMS thin film 111.

(Step S60) Next, as shown in the partial cross-sectional view shown in FIG. 1(e), in a state where the surface (also referred to as protrusion surface) of the mold 110 (for example, PDMS mold) provided with the protrusions is in contact with the resin plate 114, heating is performed while a force is applied to the mold 110 in the direction toward the resin plate 114 (in the direction indicated by arrows A2 and A3 in FIG. 1(e)) (here, as an example, while the mold is pressed from the surface opposite to the surface on which the protrusions are provided) (for example, hot pressing is performed at a predetermined temperature exceeding the glass transition point of the mold 110 and the glass transition point of the resin plate 114). Here, the resin plate 114 is made of a thermoplastic resin. Examples of the thermoplastic resin include: polyolefin resins such as polyethylene (PE), polypropylene (PP), poly(cycloolefin), and ethylene-α-olefin copolymer (for example, an ethylene-propylene copolymer); polystyrene resins such as polystyrene, styrene-butadiene-styrene block copolymer (SBS), styrene-ethylene-butylene-styrene block copolymer (SEBS), styrene-isoprene-styrene block copolymer (SIS), styrene-ethylene-propylene-styrene block copolymer (SEPS), and hydrogenated styrene-butadiene random copolymer (HSBR); polyester resins such as polybutylene terephthalate, polyethylene terephthalate, and polyethylene naphthalate; polyamide resins such as nylon 6, nylon 66, and nylon 46; acrylic resins such as polymethyl methacrylate (PMMA); polycarbonate resins; polyvinyl chloride resins; and polyvinylidene chloride resins. These can be used alone or in combination of two or more kinds thereof. The resin plate 114 is provided on a glass plate 113 as an example of a support.

As a result, as in the partial cross-sectional view shown in FIG. 1(f), the protrusion of the PDMS mold is deformed and the tip of the protrusion becomes flat. Accordingly, the bottom of the recess (also referred to as a well) formed in the resin plate becomes flat, and the thickness between the bottom surface of the recess (well) and the bottom surface of the resin plate (hereinafter, this thickness is referred to as the bottom thickness of the recess (well)) can be reduced.

Here, the resin plate is made of a resin whose temperature at which deformation occurs due to heat is lower than the heat resistant temperature of the mold, and specifically, the glass transition point when the resin plate is made of an amorphous plastic or the melting point when the resin plate is made of a crystalline plastic is lower than a heat resistant temperature of the elastic body (for example, PDMS) of the mold. In the step of processing the resin plate, the resin softened by heat is sufficiently softer than the protrusion of the mold at the initial stage of the step, and therefore the resin is deformed by being pressed against the mold, whereby a recess is formed. When the thickness of the resin is sufficient, the force required to reduce the thickness of the bottom of the recess is mainly a force required to deform the resin. On the other hand, as the step proceeds and the thin portion at the bottom of the recess becomes thinner, the force required to further reduce the thickness of the bottom of the recess is mainly a force required for discharging the resin from the thin portion against the frictional force acting between the mold or the support and the resin, rather than the force required to deform the resin. This force increases dramatically as the thickness of the thin portion decreases. In addition, since the thin portion is less likely to be deformed, the stress applied to the mold increases, and the protrusion of the mold formed of the elastic body is crushed and deformed thereby expanding the area of the thin portion. At this time, the thin portion has a nearly flat shape. Since the frictional force acting between the resin and the mold or the support further increases due to the increase in the area of the thin portion, as the thin portion of the bottom of the recess becomes thinner, the force required to further reduce the thickness of the bottom of the depression increases at an accelerated rate. Therefore, even if an error occurs in the pressure to the thin portion at each point of a workpiece due to the difference in the shape of each protrusion of the mold and the non-precision of the pressurization process, this pressure error is a very small force with respect to the pressure required to change the thickness of the thin portion that has become sufficiently thin. This allows a shape in which a recess has a thin wall to be easily and inexpensively molded, and allows a structure in which a recess has a thin wall to be molded over a wide area with a small error.

(Step S70) Next, the mold 110 is removed. Thereby, as shown in the partial cross-sectional view on the upper side in FIG. 1(g), a well plate 120 (for example, a multi-well plate) in which a recess (well) has a flat bottom and has a small bottom thickness can be produced (see the perspective view on the lower side in FIG. 1(g)). More specifically, a well plate having 384 wells in which each recess (well) has a small bottom thickness of about 10 μm can be produced.

The present embodiment is described with reference to an example in which a resin is used as the material of the well plate, but the material is not limited to this, and a metal having a temperature at which the metal is deformed by heat lower than the heat resistant temperature of the mold may be used. Specifically, the melting point of the metal may be lower than the heat resistant temperature of the elastic body of the mold. With this configuration, the metal softened by heat is sufficiently softer than the protrusion of the mold at the initial stage of the step, and therefore the metal is deformed by being pressed against the mold, whereby a recess is formed. When the thickness of the metal is sufficient, the force required to reduce the thickness of the bottom of the recess is mainly a force required to deform the metal. On the other hand, as the step proceeds and the thin portion at the bottom of the recess becomes thinner, the force required to further reduce the thickness of the bottom of the recess is mainly a force required for discharging the metal from the thin portion against the frictional force acting between the mold or the support and the metal, rather than the force required to deform the metal. This force increases dramatically as the thickness of the thin portion decreases. In addition, since the thin portion is less likely to be deformed, the stress applied to the mold increases, and the protrusion of the mold formed of the elastic body is crushed and deformed thereby expanding the area of the thin portion. At this time, the thin portion has a nearly flat shape. Since the frictional force acting between the metal and the mold or the support further increases due to the increase in the area of the thin portion, as the thin portion of the bottom of the recess becomes thinner, the force required to further reduce the thickness of the bottom of the depression increases at an accelerated rate. Therefore, even if an error occurs in the pressure to the thin portion at each point of a workpiece due to the difference in the shape of each protrusion of the mold and the non-precision of the pressurization process, this pressure error is a very small force with respect to the pressure required to change the thickness of the thin portion that has become sufficiently thin. As a result, it is possible to produce a thin-walled molded article in which a recess has a flat bottom and has a small bottom thickness (has a thin bottom wall).

A method for producing a thin-walled molded article according to the present embodiment includes: a step of heating a resin or a metal in a state of being interposed between a mold and a support in such a manner that a force in a direction to the resin or the metal is applied to the mold, wherein the mold is provided with a protrusion formed of an elastic body having a heat resistant temperature higher than a softening temperature of the resin or the metal, and the support is harder than the mold and is softened by heat at a temperature higher than a temperature for the resin or the metal; and a step of removing the mold.

With this configuration, the resin or the metal softened by heat is sufficiently softer than the protrusion of the mold at the initial stage of the step, and therefore the resin or the metal is deformed by being pressed against the mold, whereby a recess is formed. When the thickness of the resin or the metal is sufficient, the force required to reduce the thickness of the bottom of the recess is mainly a force required to deform the resin or the metal. On the other hand, as the step proceeds and the thin portion at the bottom of the recess becomes thinner, the force required to further reduce the thickness of the bottom of the recess is mainly a force required for discharging the resin or the metal from the thin portion against the frictional force acting between the mold or the support and the resin or the metal, rather than the force required to deform the resin or the metal. This force increases dramatically as the thickness of the thin portion decreases. In addition, since the thin portion is less likely to be deformed, the stress applied to the mold increases, and the protrusion of the mold formed of the elastic body is crushed and deformed thereby expanding the area of the thin portion. At this time, the thin portion has a nearly flat shape. Since the frictional force acting between the resin or the metal and the mold or the support further increases due to the increase in the area of the thin portion, as the thin portion of the bottom of the recess becomes thinner, the force required to further reduce the thickness of the bottom of the depression increases at an accelerated rate. Therefore, even if an error occurs in the pressure to the thin portion at each point of a workpiece due to the difference in the shape of each protrusion of the mold and the non-precision of the pressurization process, this pressure error is a very small force with respect to the pressure required to change the thickness of the thin portion that has become sufficiently thin. This allows a shape in which a recess has a thin wall to be easily and inexpensively molded, and allows a structure in which a recess has a thin wall to be molded over a wide area with a small error. As a result, it is possible to produce a thin-walled molded article in which a recess has a flat bottom and has a small bottom thickness (has a thin bottom wall). This makes it possible to produce a thin-walled molded article (for example, a well plate) in which a recess has a flat bottom and has a small bottom thickness (has a thin bottom wall). This allows a shape in which a recess has a thin wall to be easily and inexpensively molded, and allows a structure in which a recess has a thin wall to be molded over a wide area with a small error. Besides, a thicker shape and a thinner shape can be integrally molded. Further, a thicker shape and a thinner shape can be continuously formed with a curved surface.

A method for producing a thin-walled molded article according to the present embodiment further includes: a step of producing a jig with a through hole, provided with a thin film of an elastic body on its front surface; a step of sucking the thin film from a back surface side through the through hole; a step of injecting a solution of an elastic body from above the thin film deflected after the sucking; and a step of heating and curing the solution to form the mold.

With this configuration, it is possible to produce a mold provided with a protrusion made of an elastic body. This mold is heated in a state in which the surface provided with the protrusion is in contact with a resin or a metal that is deformed by heat at a temperature lower than a heat resistant temperature of the mold, in such a manner that a force in a direction toward the resin is applied to the mold, whereby a thin-walled molded article (for example, a well plate) in which a recess has a flat bottom and has a small bottom thickness can be produced.

### Example 1

Hereinafter, methods of respective steps according to Example 1 are described.

<Exemplary Method for Producing PDMS Thin Film>

Here, an exemplary method for producing a PDMS thin film in step S10 of FIG. 2 is described. A PDMS solution was dropped onto a polyethylene naphthalate (PEN) film cut out into a first size, and after being left to stand for a certain period of time so that air bubbles are removed, the film was spin-coated at a predetermined rotation speed for a first set time using a spin coater. This was placed on a tempered glass (for example, Tempax) of a second size larger than the first size and heated at a prescribed temperature higher than room temperature for a second set time. The PDMS thin film was taken out together with the tempered glass (for example, Tempax) and heat was dissipated in a clean booth. Two double-sided tapes for silicone rubber were stuck on a frame portion of a hollow acrylic frame, and this was pressed against the PDMS thin film so that the acrylic frame and the PDMS thin film are bonded to each other with the double-sided tapes. The PDMS thin film is peeled off from the PEN film using the acrylic frame as a supporting substrate. As a result, a PDMS thin film-attached frame (see FIG. 3) in which the PDMS thin film is provided in the hollow portion of the acrylic frame is produced.

<Method for Producing Jig Having Through Holes in which PDMS Thin Film is Provided on Front Surface>

A method for producing a jig having a through holes, provided with a PDMS thin film on its front surface, is described with reference to FIG. 3. FIG. 3 is an exploded perspective view for explaining attachment of a PDMS thin film to a jig having through holes. In FIG. 3, a PDMS thin film-attached frame 11 provided with a PDMS thin film 111 is produced by the above-described method for producing a PDMS thin film.

A well plate processing member 12, the adhesive is removed from the bottom surface of the bottomless-well plate (here, as an example, a plate of 384 bottomless wells), and the bottom surface side is set to the upper side (the PDMS thin film-attached frame 11 side) at the time of attachment. The well plate processing member 12 has a bottom surface-side outer peripheral frame portion cut by a predetermined length (for example, being planed after being processed with an ultrasonic cutter), so as to be formed as a portion into which the PDMS thin film-attached acrylic frame is fitted. A packing 17 using a silicone adhesive and a polypropylene plate is provided on a contact surface with a plate 14 having a plurality of through holes described later.

A height adjustment shim plate 13 has such a configuration that a shim plate is placed on the outer peripheral frame portion of the well plate processing member and interposed between the outer peripheral frame portion and the PDMS thin film-attached frame 11 so that the well portion of the well plate processing member 12 and the PDMS thin film-attached frame 11 have substantially identical heights.

The plate 14 having a plurality of through holes is, for example, an acrylic plate in which a plurality of (for example, 384) through holes are provided at intervals.

A stage 15 is, for example, an aluminum stage. The stage 15 is placed in a die cast box 16 described later and supported from below so that the plate 14 having a plurality of through holes is not distorted at the time of suction.

The die cast box 16 is a die cast box made of aluminum. The die cast box 16 includes a rectangular bottom plate and four side plates connected to respective sides of the bottom plate. One side plate of the die cast box 16 is provided with a through hole by thread cutting, and a hollow member 18 (for example, a tube joint) is attached to the through hole. The hollow member 18 serves as an inlet port for suction. The die cast box 16 has an open upper surface, and a packing 19 is provided at an edge on the upper surface side of each of the four side plates. As a result, the packing 17 and the packing 19 are in air tight contact with each other, so that the space between the bottom surface of the well plate processing member 12 and the die cast box 16 can be sealed to prevent air leakage.

The components illustrated in FIG. 3 are assembled as illustrated in FIG. 3, whereby a jig having through holes in which a PDMS thin film is provided on the front surface is produced. Then, air is sucked through the hollow member 18, whereby the PDMS thin film is sucked.

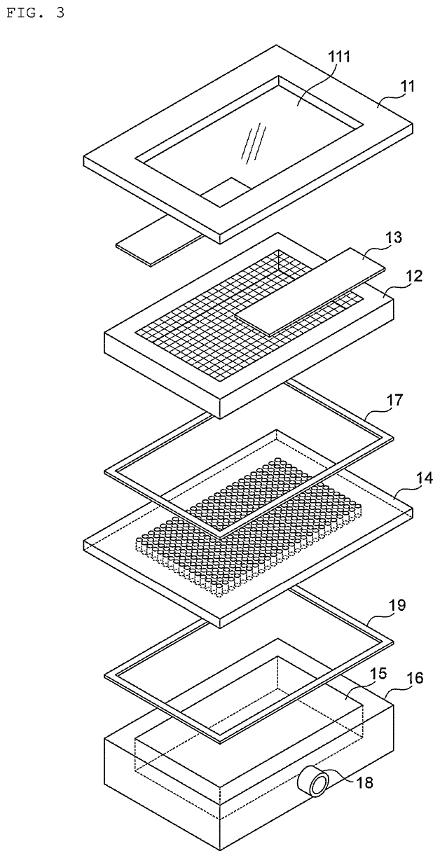

The relationship between the suction pressure and the distance by which the center of the PDMS thin film changes due to suction is described with reference to FIG. 4. FIG. 4 is a graph showing exemplary experimental results regarding the relationship between the suction pressure and the change distance of the center of the PDMS thin film. The vertical axis represents the change distance of the center of the PDMS thin film, and the horizontal axis represents the suction pressure. FIG. 4 shows the results of measuring the change distance of the center of the PDMS thin film using a confocal microscope while changing the suction pressure. As shown in FIG. 4, the change distance of the center of the PDMS thin film increased as the suction pressure increased. As shown in FIG. 4, by changing the suction pressure, a mold having protrusions of a desired height can be produced, so that a well plate of a desired depth can be produced. The measurement was performed by the following method.

<Method for Experiment of Measuring Change Distance of Center of PDMS Thin Film>

The PDMS mold producing system of FIG. 3 was set upside down on the stage of a confocal microscope. At that time, the upper optical system (halogen lamp and the like) of the confocal microscope was tilted backward, and a safety switch at the base of the upper optical system was maintained in a state of being pressed always by a silicone plate. A diaphragm pump and a regulator were connected to a jig set for PDMS mold production, and suction was performed at −0 to −0.06 mPa. The 3D shape of the PDMS thin film at that time was observed in ZStack mode. The change distance of the center of the film due to suction was determined from the difference between the Z coordinates of the outside and the center of the well, the Z coordinates being measured by detection of reflection of a 488 nm laser (n=4 wells, excitation wavelength: 488 nm, fluorescence wavelength: 483 to 493 nm). In addition, the film thickness was determined by a similar method from a difference between Z coordinates of the upper and lower surfaces of the film (n=4 wells, excitation wavelength: 488 nm, fluorescence wavelength: 483 to 493 nm).

The change distance of the center of the PDMS thin film is 1664.7±12.5 μm (n=4 wells) when suction was performed at 0.027 mPa. As an example, this pressure of 0.027 mPa was assumed to be a set pressure at the time of suction in this mold production. The thickness of the PDMS thin film was 46.3±0.7 μm (n=4 wells).

<Method for Producing PDMS Mold>

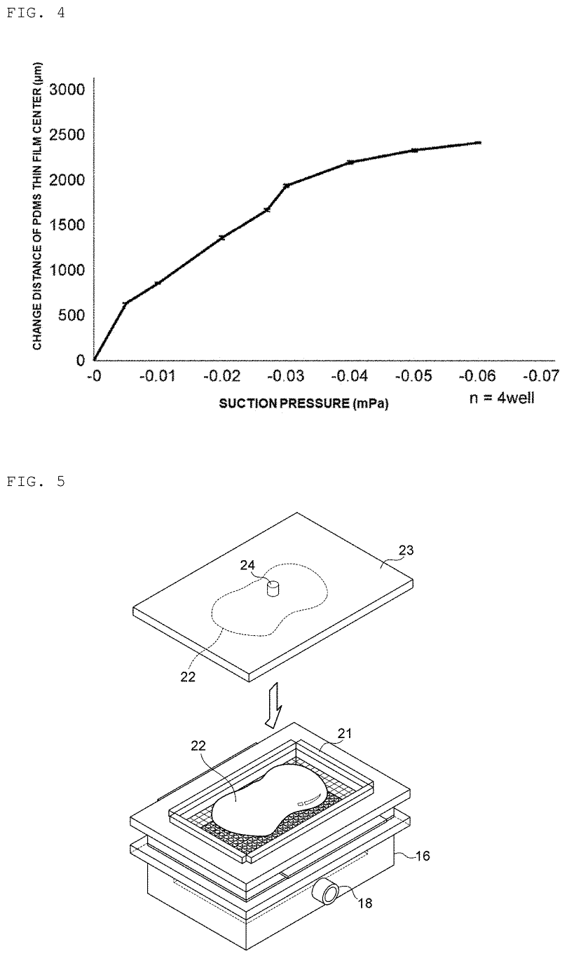

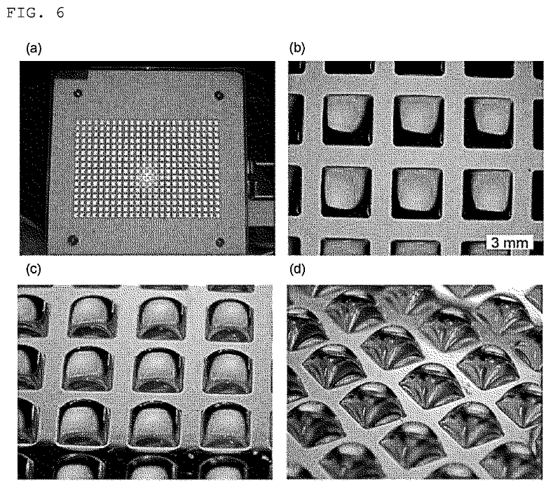

Next, an exemplary method for producing a PDMS mold is described with reference to FIG. 5. FIG. 5 is a schematic diagram for explaining a method for producing a PDMS mold. As shown in FIG. 5, a mold having about 1.65 mm-high protrusions was produced with PDMS using the PDMS mold producing system of FIG. 3.

In the PDMS mold producing system, silicone rubber banks 21 were placed on four sides to the well plate processing member 12 on the PDMS thin film so as to surround the well plate processing member 12. A predetermined amount of a PDMS solution was dropped from above, and nitrogen gas was blown to spread the PDMS solution so as to cover the wells. This was placed in a desiccator and defoamed under reduced pressure. A diaphragm pump and a regulator were connected to the hollow member 18 in the PDMS mold producing system, and the pressure reduction of −0.027 mPa was performed. A specified amount of the PDMS solution 22 was dropped to a tempered glass 23 (for example, Tempax), and with this as a starting point, the tempered glass 23 and the PDMS solution 22 on the PDMS thin film were brought into contact with each other, whereby the tempered glass 23 was covered so that air bubbles did not enter. This was placed on a lower stage of a heat press machine at a first temperature higher than room temperature, and by raising its jack, it was pressed with an upper stage with a cylindrical rubber 24 connected to the center of the tempered glass 23 being used as a cushion. The upper and lower stages of the heat press machine were covered with aluminum foil, and kept warm. After heating and curing for a specified time, the PDMS mold producing system was decomposed to take out the PDMS mold (PDMS+Tempax), and the PDMS was completely cured by further heating at a second temperature higher than the first temperature for a predetermined time.

FIG. 6(a) is an overall image of a PDMS mold photographed from above with a stereomicroscope. FIG. 6(b) is a magnified image of the PDMS mold photographed from above with a stereomicroscope. FIG. 6(c) is a magnified image of the PDMS mold photographed at a diagonal angle from above with a stereomicroscope. FIG. 6(d) is a magnified image of the PDMS mold photographed at another diagonal angle from above with a stereomicroscope. As a result of the production of the PDMS mold, as shown in FIG. 6(d), a PDMS mold in which 384 arcuate protrusions having smooth vertices were arranged in an array was produced. As a result of measurement by the following measurement method using a confocal microscope, the protrusion height was 1650.3±7.2 μm (n=4 wells, mean±standard deviation), and it was confirmed that a mold having a shape with a height close to the target (about 1650 μm) was produced. Here, the protrusion height represents the average±standard deviation of the protrusion heights of four wells located at the center of each plate.

<Method of Measuring Protrusion Height>

The appearance of the produced PDMS mold was photographed and observed with a stereomicroscope. In addition, the PDMS mold was placed on a confocal microscope stage with the protrusions facing down, the Z coordinates of the outside of the wells and the tips of the protrusions (also referred to as projections) were measured by detection of reflection of a 488 nm laser, and the projection height of the PDMS mold was determined from the differences (n=4 wells, excitation wavelength: 488 nm, fluorescence wavelength: 483 to 493 nm). In addition, a 3D shape was confirmed by autofluorescence of the PDMS mold (excitation wavelength: 405 nm, fluorescence wavelength: 435 to 445 nm).

In order to confirm the molding accuracy of the entire mold, all 384 wells of one well plate were observed with a confocal microscope in a direction perpendicular to the well bottoms, and the heights of all protrusions were measured (n=384 wells, excitation wavelength: 488 nm, fluorescence wavelength: 483 to 493 nm). As a result, the average protrusion height was 1623.5±26.5 μm (n=384 wells: average±standard deviation).

<Well Plate Molding Method>

Using a mold having a protrusion height of about 1.65 mm as an example, hot press molding of polycarbonate was performed, observation of the appearance of a molded product formed, and analysis of the 3D shape of each well using a confocal microscope were performed.

A resin plate (for example, a polycarbonate plate) was placed on a tempered glass (for example, Tempax) coated with PDMS for release, and a PDMS mold was placed thereon. Then this was set on a press stage heated to a first predetermined temperature, preheated for a first set time, and then pressed with a predetermined force for a second set time longer than the first set time. After the pressing was completed, the mold was cooled with water, and the pressure was released at the time when the temperature reached a second predetermined temperature lower than the first predetermined temperature, and the molded product was taken out. The appearance of the molded product was photographed and observed with a stereomicroscope. Using a confocal microscope, observation of broken states of the bottoms of 384 wells (n=384 well, bright field), thickness measurement and cross-sectional observation of the bottoms (n=384 well, excitation wavelength: 488 nm, fluorescence wavelength: 483 to 493 nm), and cross-sectional confirmation of the entire molded product (excitation wavelength: 488 nm, fluorescence wavelength: 483 to 493 nm) were performed.

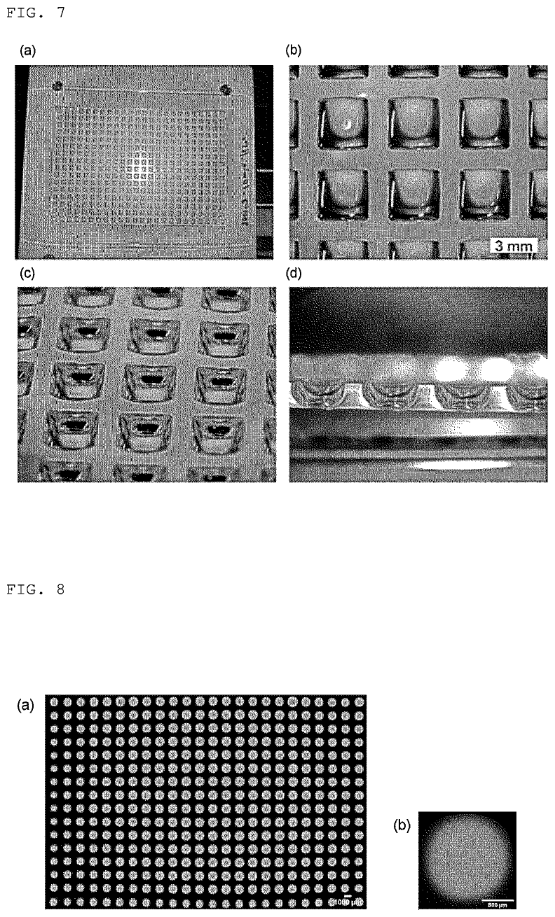

As a result, wells in each of which the thick portion forming the side wall and the thin portion forming the bottom were connected in a smooth arc shape were formed. FIG. 8(a) is an overall image of bright field observation of well bottoms according to Example 1 using a microscope. FIG. 8(b) is a magnified image of bright field observation of a well bottom of one well according to Example 1 using a microscope. In addition, as shown in FIG. 8(a) and FIG. 8(b), only interference fringes generated by the thinness were observed on the well bottoms, and no serious damage such as breakage or penetration was confirmed in any well.

FIG. 9(a) is a graph showing the distribution of protrusion heights of the PDMS mold according to Example 1. FIG. 9(b) is a graph showing the distribution of well bottom thicknesses of the molded product. FIG. 9(c) is a graph showing the distribution of well depths of the molded product according to Example 1.

As shown in FIG. 9(a), the projection heights of the PDMS mold were 1623.5±26.5 μm (n=384 wells, mean±standard deviation), and as shown in FIG. 9(b), the thicknesses of the bottom walls of the molded product were 11.9±2.4 μm (n=384 wells, mean±standard deviation). The PDMS mold had a relatively large standard deviation of 26.5 μm (difference of about 300 μm at maximum), but regarding the well bottom thickness of the molded product, the standard deviation was reduced to 2.43 μm (difference of about 14.2 μm at maximum), which was 1/10 or less. As shown in FIG. 9(c), the depths of the wells of the molded product were 1565.2±21.1 μm.

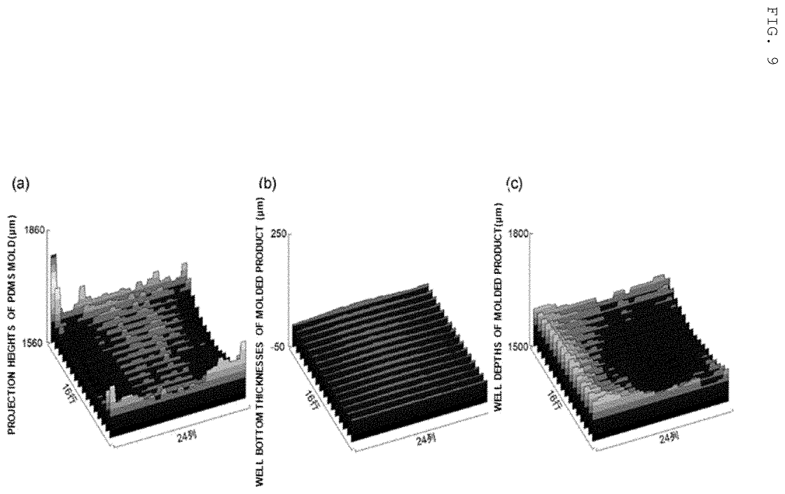

FIG. 10(a) is a cross-sectional observation image of a protrusion of the PDMS mold according to Example 1. FIG. 10(b) is a cross-sectional observation image of an entire molded product according to Example 1. FIG. 10(c) is a cross-sectional observation image of a well bottom of the molded product according to Example 1. As is clear from comparison between FIG. 10(a) and FIG. 10(b), the bottom portion was flat as compared with that of the mold, and as shown in FIG. 10(c), the bottom portion had a flat surface with a height error within 10 μm in a range of about 1000 μm in diameter. As described above, by using the present technology, errors of the thin portion of the molded product are corrected by the flexible mold, and precision processing for a wall thickness of about 10 μm can be performed even in a vast range without making a hole in the bottom wall. In addition, it has been confirmed that a very thin structure can be connected with a thick portion with a curved surface without any joint.

### Example 2

In Example 2, as is case with Example 1, a mold having protrusions (projections) in an array of 24 columns×16 rows by PDMS was produced, and the mold was pressed against a polystyrene resin plate under heating to produce a container having 384 wells each having a bottom thickness of about 25 μm.

<Production of PDMS Mold>

First, a PDMS mold having a protrusion (projection) height of about 1.85 mm was produced, and the 3D shape of the produced PDMS mold was analyzed using a confocal microscope. As a result, the average protrusion (projection) height of the PDMS mold was 1835.1±22.9 μm (n=384 wells).

<Molding of Polystyrene Well Plate>

Using the produced PDMS mold having a protrusion (projection) height of about 1.85 mm, the PDMS mold was subjected to hot press molding by pressing the protrusion-provided surface against a polystyrene plate in the same manner as in Example 1.

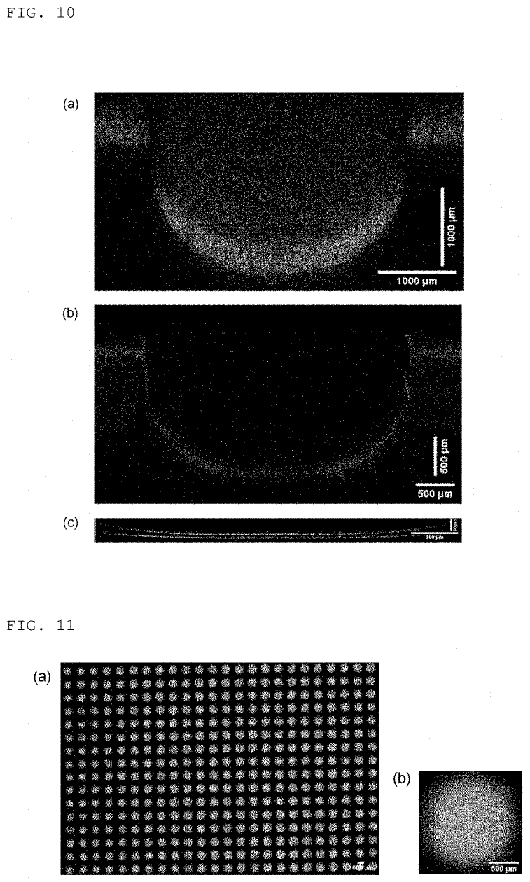

FIG. 11(a) is an overall image of bright field observation of well bottoms according to Example 2 using a microscope. FIG. 11(b) is a magnified image of bright field observation of a well bottom of one well according to Example 2 using a microscope. As confirmed in FIG. 11(a) and FIG. 11(b), no serious damage such as breakage or penetration was confirmed in any of the wells.

FIG. 12(a) is a graph showing the distribution of protrusion heights of the PDMS mold according to Example 2. FIG. 12(b) is a graph showing the distribution of well bottom thicknesses of the molded product according to Example 2. FIG. 12(c) is a graph showing the distribution of well depths of the molded product according to Example 2.

As illustrated in FIG. 12(a), the protrusions (projections) of the PDMS mold had substantially uniform heights. As shown in FIG. 12(b), the molded product had a uniform bottom thickness, and the bottom thickness of the molded product was 27.4±2.9 μm (n=384 wells). As confirmed, the PDMS mold had a standard deviation of 22.9 μm (difference of about 150 μm at maximum), but regarding the well bottom thickness of the molded product, the standard deviation was reduced to 2.9 μm (difference of about 18.3 μm at maximum), which was about ⅛. The depths of the wells of the molded product were 1612.3±15.2 μm.

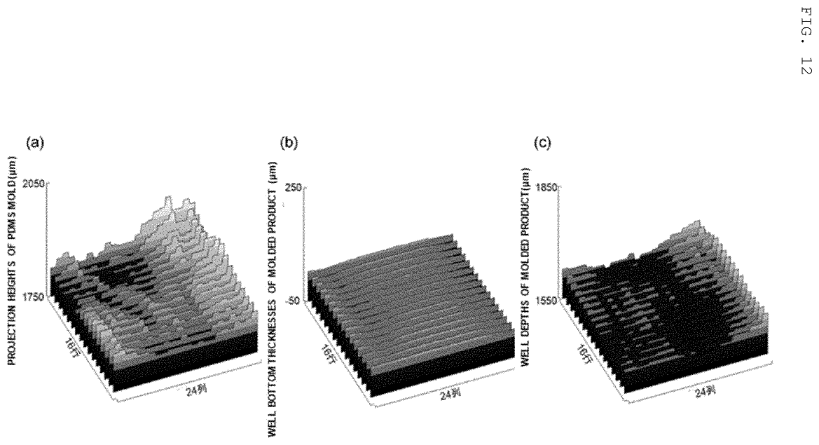

FIG. 13 is a cross-sectional observation image of a well bottom of the molded product according to Example 2. As shown in FIG. 13, the well of the molded product had a flat bottom having a height-direction error within 10 μm in a range of approximately 900 μm in diameter. Thus, it was proved that the present method can be applied to a wide range of resins as a material of a molded product.

As described above, according to the present embodiment, a thin shape can be easily and inexpensively molded. In addition, a thin structure can be molded over a wide area with a small error. Besides, a thicker shape and a thinner shape can be integrally molded. Further, a thicker shape and a thinner shape can be continuously formed with a curved surface. In addition, in the field of press molding, a technique capable of forming an extremely thin structure having a wall thickness on the order of 10 μm has not been reported, and the present technique is also excellent in precision.

The method for producing a mold in the present embodiment is an example, and the method is not limited thereto.

### Second Embodiment

Next, a second embodiment is described. In the second embodiment, the structure of the well plate produced by the production method according to the first embodiment is described.

Conventionally, there has been no well plate in which the wells had round bottoms and the bottom central portion of each well had a thickness of 200 μm or less. On the other hand, a well plate according to the second embodiment provides a well plate having wells each of which has a round bottom in which the well bottom thinnest portion has a thickness of about 200 μm or less. The well plate according to the second embodiment is made of resin, and is provided with at least one well. The well has a round bottom. The round bottom of the well allows cells to gather at the center of the bottom of the well.

<Background: Importance of High Magnification Observation>

In previous cell studies, cells have been treated as a population, and a cell population of a well unit or a dish unit has been used as one sample for various studies. For example, in cell analysis for understanding cells and biological phenomena, a method for understanding cell functions by measuring an average value of cell populations has been used. A general multi-well plate is produced for the purpose of observing an overall image of such a cell population composed of many cells or performing a cell assay or the like on rough properties of the cell population using a plate reader or the like.

However, in recent years, it has been found that the cell population, which has been considered uniform, includes an extremely small number of important cells such as stem cells and circulating tumor cells. In addition, research for analyzing individual cells such as epigenetics in detail has been reported to clarify that respective cells have differences in the molecular level, such as chemical modification of histone, and have different phenotypes and functions such as cell cycles and protein expression levels. Furthermore, researches, therapies, and the like that require precise analysis of individual cells, such as research on intratumoral heterogeneity, and elucidation of cancer therapies and immune mechanisms using immune cells typified by CAR-T cell therapy and TCR-T cell therapy, have become very important themes in medicine and life science. In the multi-well plate, a small number of cells seeded in a well is collected near the center by its round bottom shape so that detection by microscopic observation or the like is made easier. Therefore, the multi-well plate is very useful in a study of precisely analyzing a small number, ranging from one, of cells respectively, which study has become important in recent years. As a detailed cell analysis method, microscopic observation using a high magnification objective lens is often performed. For example, in a clinical sample test, the shape and the like of the nucleus in the cell are observed using an objective lens of 40 times, 63 times, or 100 times magnification. In addition, in a FISH test for detecting a specific sequence in a nucleus or a method for staining an intracellular organelle or a specific molecule with a fluorescent dye or the like and observing the same, an objective lens of a 63 times or 100 times magnification is used. Further, an objective lens of 100 times magnification is used for detailed observation of minute structures and three-dimensional detailed observation, and for example, in observation with an ultra-resolution microscopy for which the Nobel prize for chemistry was won in 2014. In addition, after such analysis of the phenotype of the cell, analysis of the genotype by PCR or the like is often performed. Therefore, in detailed microscopic analysis of a small number, ranging from one, of cells in which the multi-well plate is used, it is desirable that an objective lens of up to 100 times magnification can be applied. Since the focal length is short in the high magnification objective lens, the well bottom thickness with which the sample can be observed is limited.

<Bottom Thickness to which High Magnification Objective Lens (100 Times) can be Applied>

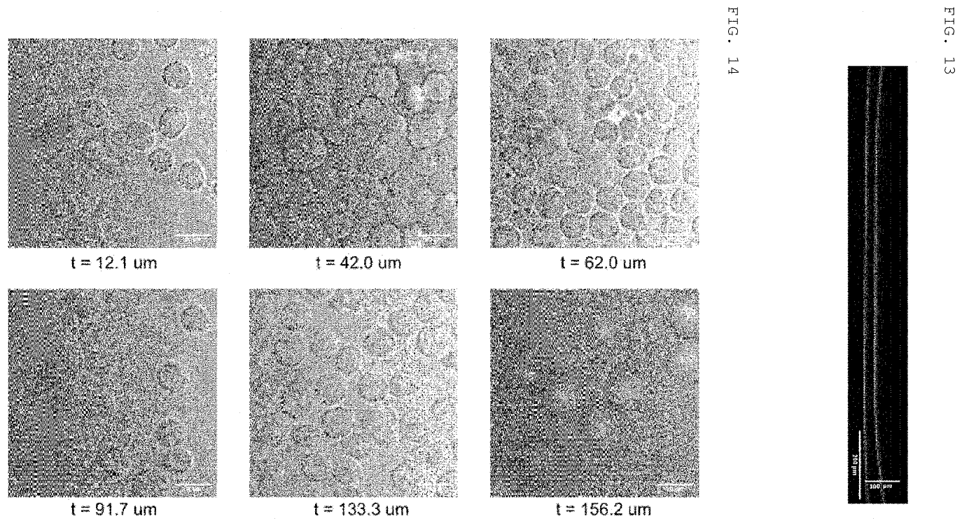

Therefore, in the present embodiment, the bottom thickness to which an objective lens of 100 times magnification can be applied is described. Using the production method according to the first embodiment, well plates provided with wells having various well bottom thicknesses and a diameter of 2 mm were produced. In this well plate, polycarbonate was used as an example of a resin used. As shown in FIG. 14, Ba/F3 cells in a well (polycarbonate) having a diameter of 2 mm were observed using an oil immersion 100× objective lens with varying well bottom thickness t.

FIG. 14 shows exemplary Ba/F3 cells imaged using an oil immersion 100× objective lens in cases of various bottom surface thicknesses. It was possible to focus on cells located on the well bottom when the bottom thickness t in the figure was up to 133.3 μm, but it was not possible to focus on cells located on the well bottom when the bottom thickness was 156.2 μm even if the oil immersion 100× objective lens was brought into contact with the well bottom. Therefore, the well bottom thickness is preferably 150 μm or less.

In this way, the well plate according to the second embodiment is preferably a well plate formed with a resin and provided with at least one well, in which the well has a round bottom, and a bottom central portion of the well has a thickness of 150 μm or less.

According to this configuration, the well has a round bottom, whereby cells seeded in the well gather at the center of the bottom of the well, which facilitates the observation of the cells. Furthermore, the thickness of bottom central portion of the well is 150 μm or less, which enables the focus to be adjusted to the cells present on the well bottom even with an oil immersion 100× objective lens, whereby detailed microscopic analysis of a small number, ranging from one, of cells can be reliably performed.

<Regulation of Lower Limit of Well Bottom Thickness>

Subsequently, the relationship between the well bottom thickness and the rate of damaged wells in 384 wells is described, which is clarified as a result of an experiment using a 384 well plate produced by the production method according to the first embodiment.

Using polycarbonate as an example of a resin, multi-well plates provided with wells having various well bottom thicknesses and a diameter of 2 mm were produced, and then the percentage of wells with characteristic damage (see FIG. 15) that increased depending on the well bottom thickness was determined. FIG. 15 is a diagram showing an example of characteristic damage that increases depending on the well bottom thinness. The image G1 is a phase image of a normal well without damage, taken by using of a 5× objective lens. On the other hand, the image G2 is a phase image of a damaged well, taken by using of a 5× objective lens. As indicated by an arrow A1 in the image G2, the well bottom had a crescent-shaped scratch. As described above, as the well bottom was thinned, a crescent-shaped scratch was more often observed on the well bottom.

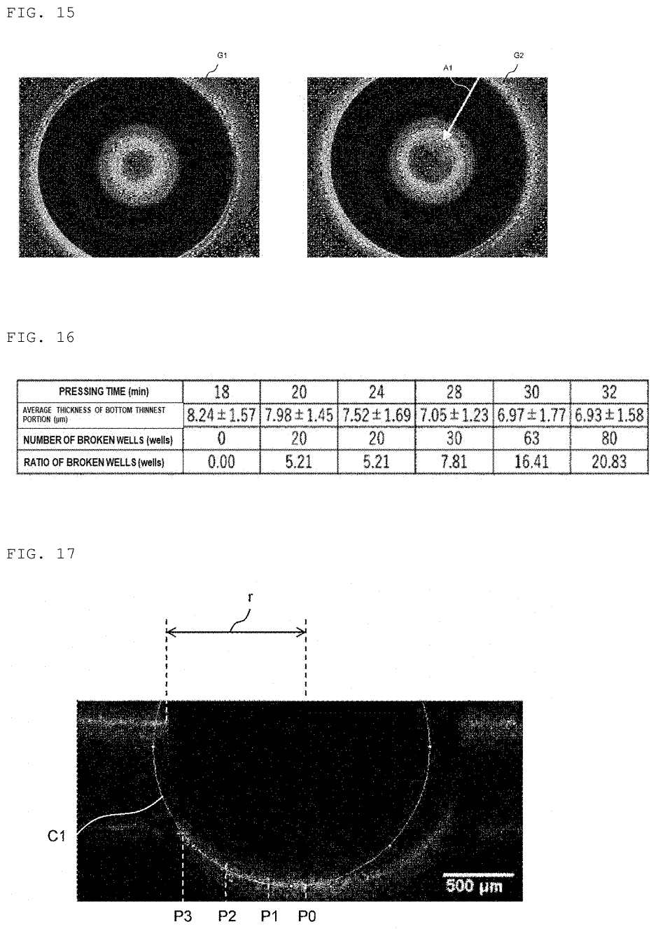

FIG. 16 is a table showing exemplary experimental results regarding the relationship between the average thickness of the well bottom thinnest portion and the damaged well rate in 384 wells. FIG. 16 shows experimental results of sets of the pressing time, the average thickness of the bottom central portion of a well (also referred to as the average bottom central portion thickness), the number of broken wells, and the ratio of broken wells. The average thickness of the well bottom thinnest portion is represented by the average value of 384 wells in the well bottom thinnest portion and standard error.

No wells with breakage were observed when the average thickness of the well bottom thinnest portion was 8.24±1.57 μm or more. When the average thickness of the well bottom thinnest portion became thinner than 7.98±1.45 μm, wells having breakage were confirmed. That is, it is critically significant that the average thickness of the bottom central portions of the wells (or the thickness of the well bottom thinnest portion) is 8 μm or more.

When the average thickness of the well bottom thinnest portion was 7.52±1.69 μm, the breakage rate was 5.21%. When the average thickness of the well bottom thinnest portion was 7.05±1.23 μm, the breakage rate was 7.81%. When the average thickness of the well bottom thinnest portion was 6.97±1.77 μm, the breakage rate was 16.81%, and when the average thickness of the well bottom thinnest portion was 6.93±1.58 μm, the breakage rate was 20.83%, which means that the breakage rate exceeded 20%. As described above, when the average thickness of the well bottom thinnest portion became less than 7 μm, the breakage rate rapidly increased. That is, it is critically significant that the average thickness of the bottom central portions of the wells (or the thickness of the well bottom thinnest portion) is 7 μm or more.

Therefore, with a view to a well plate having such a strength that most (specifically, for example, 80 to 90% or more) of the wells cannot be damaged, the average thickness of the well bottom thinnest portion (or the thickness of the well bottom thinnest portion) is preferably 7 μm or more. Therefore, with a view to a plate having such a sufficient strength that all of the wells cannot be damaged in production or the like, the average thickness of the well bottom thinnest portion (or the thickness of the well bottom thinnest portion) is preferably 8 μm or more.

<Relationship Between Thickness of Well Bottom Thinnest Portion and Curvature of Well Bottom>

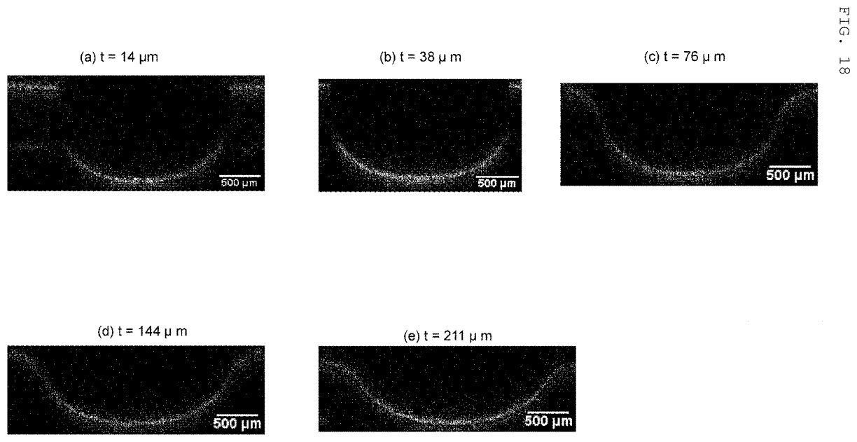

Subsequently, the well surface was coated with a 1% BSA-TAMRA/PBS solution overnight, and then fluorescence of BSA-TAMRA on the well surface was observed with a confocal microscope. FIG. 17 is an exemplary image of a longitudinal section of a well obtained by a confocal microscope. Shown are points P1, P2, and P3 on the well bottom that are located at distances in the horizontal direction from the center P0 of the well bottom, the distances being 30% (0.3 mm as an example), 60% (0.6 mm as an example), and 90% (0.9 mm as an example), respectively, of the radius r (1 mm as an example here) of the well. Here, the radius of the well is a radius of a circle indicated by a horizontal cross-section of the well at a height above the well bottom by 80% of the height from the well bottom to the opening; however, when, above the well bottom, there is no well wall in which the tangent of the well wall in the vertical cross section of the well is 80° to 90° with respect to the horizontal plane in the range of 80% to 100% of the height from the well bottom to the opening, the radius of the well is a radius of a circle indicated by the horizontal cross section of the well at the height above the well bottom by 50% of the height from the well bottom to the opening. A circle Cl passing through the points P1, P2, and P3 is illustrated. In the present embodiment, the radius of the circle Cl is defined as the radius of curvature of the well bottom.

FIG. 18 shows exemplary images of longitudinal sections of wells obtained by a confocal microscope in cases of various bottom surface thicknesses. FIG. 18(a) is an exemplary image of a longitudinal cross section of a well in a case where the thickness t of the well bottom thinnest portion is 14 μm, FIG. 18(b) is an exemplary image of a longitudinal cross section of a well in a case where the thickness t of the well bottom thinnest portion is 38 μm, FIG. 18(c) is an exemplary image of a longitudinal cross section of a well in a case where the thickness t of the well bottom thinnest portion is 76 μm, FIG. 18(d) is an exemplary image of a longitudinal cross section of a well in a case where the thickness t of the well bottom thinnest portion is 144 μm, and FIG. 18(e) is an exemplary image of a longitudinal cross section of a well in a case where the thickness t of the well bottom thinnest portion is 211 μm. In this way, when the thickness t of the well bottom thinnest portion changes, the shape of the well bottom changes, and the curvature of the well bottom changes.

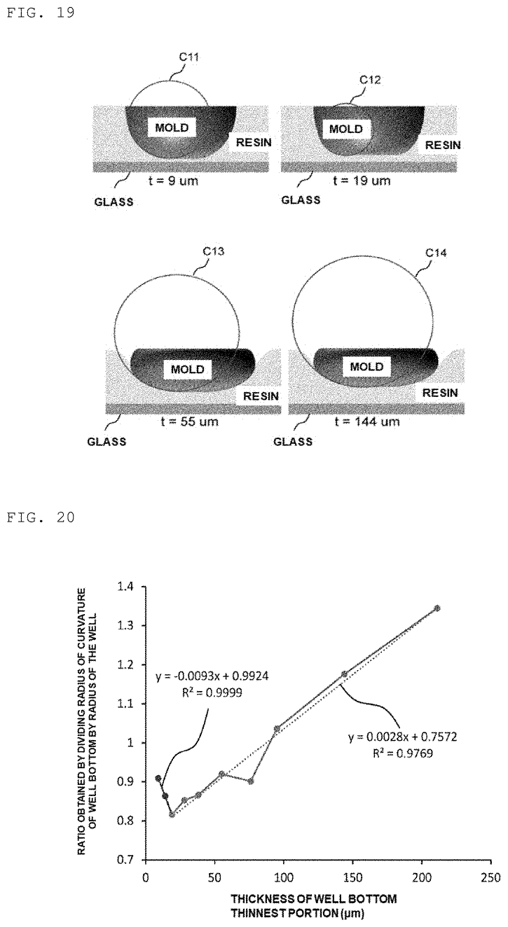

FIG. 19 is a schematic diagram for explaining a difference in the radius of curvature due to a difference in the thickness t of the well bottom thinnest portion. The shape of the well obtained from the experimental results of FIG. 18 is qualitatively described using FIG. 19. The radius of the well is 1 mm. When the thickness t of the well bottom thinnest portion was 9 μm, the radius of curvature was 0.91 mm, which is smaller than 1 mm when the mold is not deformed, as represented by a circle C11 of the radius of curvature. In addition, when the thickness t of the well bottom thinnest portion was about 19 μm, for example, the radius of curvature was 0.82 mm, which was the smallest, as represented by a circle C12 of the radius of curvature. This is considered to be because, as a principle of the present resin processing method, the mold bottom portion close to the underlying glass plate receives particularly large stress, and thus the mold bottom portion is greatly deformed as compared with other portions of the mold. When the thickness t of the well bottom thinnest portion was 55 μm, the radius of curvature was 0.92 mm, which is smaller than 1 mm when the mold is not deformed, as represented by a circle C13 of the radius of curvature.

On the other hand, when the thickness t of the well bottom thinnest portion increases to about 76 μm or more, the action of the mold bottom portion receiving a particularly large stress decreases, and the entire mold is uniformly stressed and deformed into a flat shape. For example, when the thickness t of the well bottom thinnest portion was 144 μm, as represented by a circle C14 of a radius of curvature in this case, the radius of curvature was 1.2 mm, and when the thickness t of the well bottom thinnest portion was 211 μm, a radius of curvature of 1.3 mm. Thus, the radius of curvature gradually increased, and exceeded the radius of curvature of 1 mm when the mold is not deformed.

FIG. 20 is a graph showing experimental results regarding the relationship between the thickness of the well bottom thinnest portion and the ratio obtained by dividing the radius of curvature of the well bottom by the radius of the well.

The well plate produced by the production method according to the present embodiment has a characteristic well bottom shape, and the relationship between the thickness of the well bottom thinnest portion and the ratio obtained by dividing the radius of curvature of the well bottom by the radius of the well has the relationship shown in the graph of FIG. 20. The radius of curvature of the well bottom used in calculating the ratio of each plot of the graph in FIG. 20 was an average of four values of the radii of curvature of the well bottom measured from arcs of bottom surfaces of four sides of the well. The four sides of the well were, for example, the sides in two directions that were opposite to each other, parallel to the vertical rows of the wells, and passing through the centers of the well bottoms, and the sides in two directions that were opposite to each other, parallel to the horizontal rows of the wells, and passing through the centers of the well bottoms. The four directions for the measurement of the radius of curvature of the well bottom may be directions perpendicular to each other.

Each plot of the graph of FIG. 20 represents a point of a representative example. When a regression equation is applied to each plot in which the bottom central portion of the well in FIG. 20 is in the range of 7 to 19 μm, the ratio obtained by dividing the radius of curvature of the well bottom by the radius of the well is given as y, and the bottom central portion of the well is given as x [μm], then, the ratio y is expressed as y=−0.0093x+0.9924 when the bottom central portion of the well is in the range of 7 to 19 μm. The coefficient of determination R² of this regression equation is 0.9999. In addition, both the points of the representative example and the points other than the representative example fall within ±10% of the value of y=−0.0093x+0.9924.

Therefore, when the bottom central portion of the well is in the range of 7 to 19 μm, the ratio y satisfies the value of y=−0.0093x+0.9924 or is within ±10% of this value.

When a regression equation is applied to each plot in which the bottom central portion of the well in FIG. 20 is in the range of 19 to 200 μm, the ratio y satisfies y=0.0028x+0.7572 when the bottom central portion of the well is in the range of 19 to 200 μm. The coefficient of determination R² of this regression equation is 0.9769. In addition, both the points of the representative example and the points other than the representative example fall within ±10% of the value of y=0.0028x+0.7572.

Therefore, when the bottom central portion of the well is in the range of 19 to 200 μm, the ratio y satisfies y=0.0028x+0.7572 or is within ±10% of this value. With this configuration, it is possible to reduce the well bottom thickness with a round bottom.

In the range where the thickness x of the well bottom thinnest portion is in a range of 0 to 200 μm for the above two equations, the ratio obtained by dividing the radius of curvature of the well bottom by the radius of the well is 0.7 to 1.5. With this configuration, it is possible to reduce the well bottom thickness with a round bottom.

The present invention is not limited to the above-described embodiment as it is, and can be embodied by modifying the components without departing from the spirit of the present invention in the implementation stage. In addition, various inventions can be formed by appropriately combining a plurality of constituent elements disclosed in the above embodiments. For example, some components may be deleted from all the components shown in the embodiments. Furthermore, constituent elements in different embodiments may be appropriately combined.

### REFERENCE SIGNS LIST

11 PDMS thin film-attached acrylic frame
12 well plate processing member
13 height adjustment shim plate
14 plate having a plurality of through holes
15 stage
16 die cast box
17, 19 packing
18 hollow member
21 silicone rubber bank
22 PDMS solution
23 tempered glass
24 rubber

1. A method for producing a thin-walled molded article, comprising:
a step of applying a force to a mold in a direction to a resin or a metal under heating condition in a state that the resin or the metal is interposed between the mold and a support, wherein the mold is provided with a protrusion formed of an elastic body having a heat resistant temperature higher than a softening temperature of the resin or the metal, and the support is harder than the mold and is softened by heat at a temperature higher than a temperature for the resin or the metal;
a step of molding the resin or the metal by the applied force to form a recess having a thin portion at the bottom, wherein, in the process of forming the recess, the protrusion of the mold is deformed to expand the area of the thin portion, and thereby the thin portion becomes a flat shape; and
a step of removing the mold.

2. The method for producing a thin-walled molded article, according to claim 1,
wherein the resin is an amorphous plastic resin having a glass transition point lower than a heat resistant temperature of the elastic body of the mold, or a crystalline plastic having a melting point lower than the heat resistant temperature of the elastic body of the mold.

3. The method for producing a thin-walled molded article, according to claim 1,
wherein the metal has a melting point lower than the heat resistant temperature of the elastic body of the mold.

4. The method for producing a thin-walled molded article, according to claim 1,
wherein the resin is a thermoplastic resin.

5. The method for producing a thin-walled molded article, according to claim 1, further comprising:
a step of producing a jig having a through hole, provided with a thin film of an elastic body on its front surface;
a step of sucking the thin film from a back surface side through the through hole;
a step of injecting a solution of an elastic body from above the thin film deflected after the sucking; and
a step of heating and curing the solution to form the mold.

6. The method for producing a thin-walled molded article, according to claim 1,
wherein the elastic body is polydimethylsiloxane.

7. The method for producing a thin-walled molded article, according to claim 1,
wherein the thin-walled molded article is a well plate.

8. A well plate that is made of resin and is provided with at least one well,
wherein the well has a round bottom, and
a bottom central portion of the well has a thickness of 200 μm or less.

9. The well plate according to claim 8,
wherein a ratio obtained by dividing a radius of curvature of a well bottom by a radius of the well is 0.7 to 1.5.

10. The well plate according to claim 8,
wherein, when a ratio obtained by dividing the radius of curvature of the well bottom by the radius of the well is given as “y”, and a bottom central portion of the well is given as “x” [μm],
when the bottom central portion of the well is in a range of 7 to 19 the ratio y satisfies y=−0.0093x+0.9924 or is within ±10% of this value, and
when the bottom central portion of the well is in a range of 19 to 200 the ratio y satisfies y=0.0028x+0.7572 or is within ±10% of this value.

11. The well plate according to claim 8,
wherein the bottom central portion of the well has an average thickness of 7 μm or more.

12. The well plate according to claim 8,
wherein the bottom central portion of the well has a thickness of 150 μm or less.

13. The method for producing a thin-walled molded article according to claim 1, wherein the standard deviation of the thickness of the bottom in the thin-walled molded article is smaller than the standard deviation of the heights of the protrusion of the mold.

14. The method for producing a thin-walled molded article according to claim 1, wherein the standard deviation of the thickness of the bottom in the thin-walled molded article is smaller than ⅛ of the standard deviation of the heights of the protrusion of the mold.

15. The method for producing a thin-walled molded article according to claim 1, wherein the elastic body is a silicone resin.
